(19) United States
(12) Patent Application Publication
(10) Pub. No.: US 2026/0183566 A1
(43) Pub. Date: Jul. 2, 2026

(54) BRACHYTHERAPY APPLICATOR

(71) Applicant: Northwestern University, Evanston, IL (US)

(72) Inventors: Shuhua ZHENG, Evanston, IL (US); Jonathan B. Strauss, Evanston, IL (US); Eric D. Donnelly, Evanston, IL (US); Jooyoung Sohn, Evanston, IL (US)

(21) Appl. No.: 19/131,059

(22) PCT Filed: Nov. 21, 2023
(86) PCT No.: PCT/US2023/080689

§ 371 Date: May 19, 2025

Related U.S. Application Data
(60) Provisional application No. 63/426,835, filed on Nov. 21, 2022.

(51) Int. Cl. A61N 5/10

(52) U.S. Cl. CPC A61N 5/1016

(57) ABSTRACT

A device with a central cylinder including multiple passageways and a plurality of channels spaced circumferentially around the central passageway. The device further includes a ring with a plurality of outlets and a plurality of frames coupled to the ring. Each of the plurality of frames includes a frame channel, a frame opening, and a distal end coupled to one of the plurality of outlets. A plurality of supports extend between the cylinder and the plurality of frames, and each of the plurality of supports is coupled to the cylinder with an adjustable lock. Each of the plurality of frames is adjustable with respect to the cylinder in response to movement of the corresponding adjustable lock with respect to the cylinder.

### RELATED APPLICATIONS

This application claims priority to and the benefit of U.S. Provisional Patent Application No. 63/426,835, filed on Nov. 21, 2022, which is incorporated herein by reference in its entirety for all purposes.

### TECHNICAL FIELD

The present disclosure relates to a novel applicator for gynecologic high-dose rate brachytherapy, and in particular for interstitial brachytherapy (ISBT).

### BACKGROUND

Gynecologic cancers, which typically include ovarian, cervical, uterine, vaginal and valvular cancers, are major causes of cancer-related death in women. Cervical cancer is the fourth leading cause of cancer death in women worldwide, with 604,000 new cases diagnosed in 2020. In the United States, there were an estimated 14,400 cases and over 4,200 women died in 2021. Endometrial cancer represents the fourth leading cause of cancer in women in the United States. In 2022 65,950 new cases of cancer are expected to be diagnosed in the U.S. alone with 12,550 estimated deaths. In addition, an estimated 8,870 new vaginal cases in the U.S. are expected to be diagnosed in 2022 with about 1,600 deaths.

Radiotherapy, including both brachytherapy and external beam radiotherapy (EBRT), play a critical role in the treatment of many gynecological malignancies. Locally advanced cervical cancer requires definitive treatment with chemotherapy, external beam radiotherapy and brachytherapy. In endometrial cancer, radiotherapy (RT) is an integral part of adjuvant therapy for patients with high-risk features including FIGO stage I, grade 3 with deep myometrial invasion or lymphovascular space invasion (LVSI), Stage II or III disease, or Stage I-III with unfavorable histological features such as clear cell or serous. For medically inoperable patient with endometrial cancer, chemoradiotherapy (chemoRT) can be considered for definitive treatment or to reduce tumor burden to make patients medically operable.

Compared with external beam radiotherapy (EBRT), brachytherapy places radioactive source in close proximity to tumor which enables a high dose of radiation delivery directly to the target and decreased dose to adjacent tissue secondary to rapid dose fall off. Intracavitary cylinder brachytherapy is mostly commonly used in the definitive treatment of locally advanced cervical cancer during which a tandem or applicator is placed within an existing cavitary (i.e., through cervix into endometrial canal). Interstitial brachytherapy (ISBT), involves insertion of needles directly into the tumor mass. ISBT is often indicated for bulky lesions, patients who have undergone a hysterectomy, lower vaginal lesions, and tumors with lateral extension to the parametria or pelvic sidewalls.

### SUMMARY

The disclosure provides, in one aspect, a device design of a cylinder which includes a central passageway and a plurality of channels spaced circumferentially around the central passageway. The device also includes a secondary frame system with a plurality of outlets and a plurality of frames coupled to the primary ring. A plurality of supports extend between the cylinder and the plurality of frames, and each of the plurality of supports is coupled to the cylinder with an adjustable lock. Each of the plurality of frames is adjustable with respect to the cylinder in response to movement of the corresponding adjustable lock with respect to the cylinder.

In some embodiments, each of the plurality of supports is coupled to one of the plurality of frames with a hinge.

In some embodiments, each of the plurality of frames is equally spaced apart circumferentially around the cylinder.

In some embodiments, each of the plurality of frames is coupled to the ring with a socket connection.

In some embodiments, the device further includes a wedge positioned at a frame opening of at least one of the plurality of frames.

In some embodiments, the device further includes a plurality of connection tubes extending between the cylinder and the ring.

In some embodiments, each of the plurality of connection tubes extending between one of the plurality of channels and one of the plurality of outlets.

In some embodiments, the cylinder defines a longitudinal axis and the adjustable lock slides along the longitudinal axis.

In some embodiments, the adjustable lock is a knob gear.

In some embodiments, each of the plurality of frames includes a frame channel, a frame opening, and a distal end coupled to one of the plurality of outlets.

The disclosure provides, in one aspect, an applicator device, a tandem, and a plurality of brachytherapy catheters.

Other aspects of the disclosure will become apparent by consideration of the detailed description and accompanying drawings.

### BRIEF DESCRIPTION OF THE DRAWINGS

These features, aspects, and advantages of the present technology will be presented in the following drawings which will aid in a better understood of the described device. The accompanying figures and examples are provided by way of illustration and not by way of limitation.

FIG. 1 is a schematic of an applicator with only two frames illustrated for clarity.

FIG. 2 is a schematic of the applicator of FIG. 1 in combination with a central tandem at least partially positioned through the applicator.

FIG. 3 is a schematic of a cross-section of an applicator.

FIG. 4 is a schematic of a connection between a frame and the ring.

FIG. 5 is a schematic of an applicator, illustrating a plurality of potential insertion points and adjustable angles for insertion as dashed arrows.

FIG. 6 is a schematic of an applicator adjusted for targeted delivery of multiple needles to a tumor.

FIG. 7 is a schematic the applicator of FIG. 2 in-use.

FIG. 8 is a perspective view of an applicator.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in a variety of manners given flexibility of design all of which could not be fully described or illustrated.

### DETAILED DESCRIPTION

All publications, patent applications, patents and other references mentioned herein are incorporated by reference in their entirety. The materials, methods, and examples disclosed herein are illustrative only and not intended to be limiting.

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. The terms “comprise(s),” “include(s),” “having,” “has,” “can,” “contain(s),” and variants thereof, as used herein, are intended to be open-ended transitional phrases, terms, or words that do not preclude the possibility of additional acts or structures.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “subject” and “patient” refer to any animal, such as a dog, cat, bird, livestock, and particularly a mammal, preferably a human.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “coupled,” as used herein, is defined as “connected,” although not necessarily directly, and not necessarily mechanically. The term coupled is to be understood to mean physically, magnetically, chemically, fluidly, electrically, or otherwise coupled, connected or linked and does not exclude the presence of intermediate elements between the coupled elements absent specific contrary language.

Placement of interstitial needles is usually carried out with applicators that serve as a guide for needle insertion. Brachytherapy needles are hollow tubes (e.g., catheters) that allow loading of radioactive sources within or near the tumor. Needle insertion is usually done in the operating room (OR) or brachytherapy suite with the patient under appropriate anesthesia or epidural sedation. Due to limitations in real time imaging availability, placement of interstitial needles is often performed without direct visualization of the needle insertion depth, angle and final anatomic location. As a result, the quality of brachytherapy needle placement is highly user dependent and poor quality of implant placement can result in decreased target dosing and increased risk of disease recurrence. Meanwhile misplacement of needles into normal organs, such as bowel and bladder can lead to significant acute and late complications (i.e. enterotomies). After placement of a conventional applicator, the vaginal canal is packed with gauze in an effort to stabilize the applicator and also displace the rectum and bladder away from the applicator. The gauze remains in place during the duration of the procedure which can cause significant discomfort, pain and potentially increase risk of vaginal infection.

Existing interstitial brachytherapy applicators have several disadvantages, including: (1) the applicator inhibiting visualization of the cervix during needle insertion; (2) the procedure requires packing of the vaginal canal (e.g., with gauze); (3) to reach distant vaginal disease needles must transverse through a perineal plate which may cause increased risk of pain and bleeding; (4) may need Smit sleeve placement which sometimes entails an operating room and/or general anesthesia and will sometimes cause significant pain post-operatively; (5) existing applicators cannot direct needles at a single location from multiple points and angles; (6) existing applicators require separate needles to treat both parametrial and pelvic wall disease the same time; (7) increased risk of possible applicator movement; and (8) existing applicators often require intra operative CT or MRI for needle placement adjustment, which has limited availability.

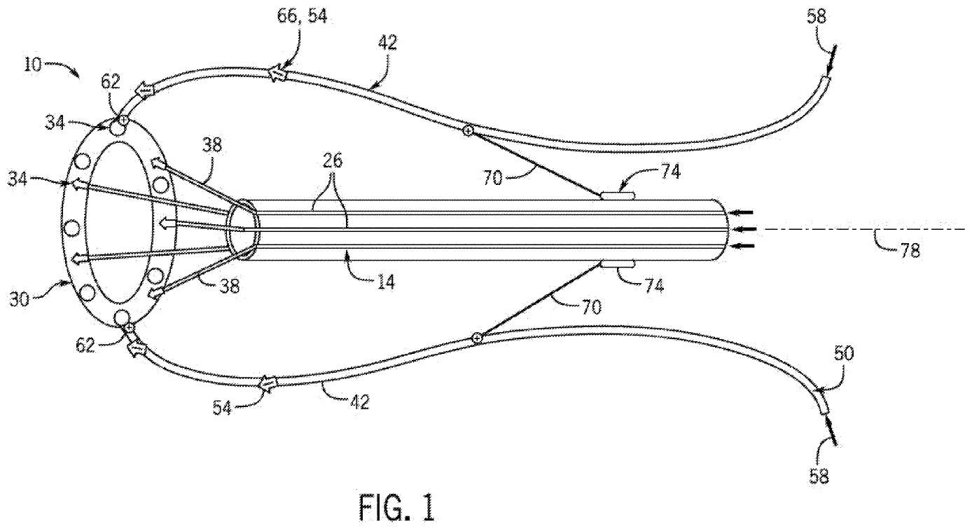

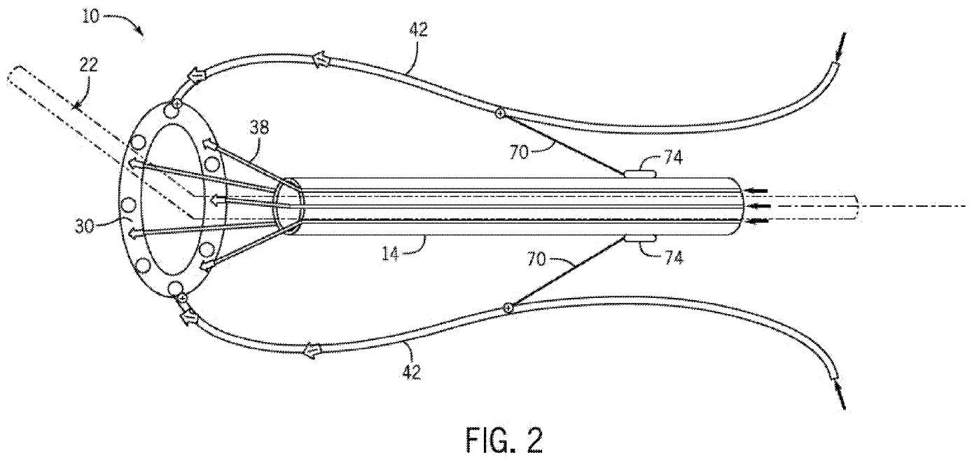

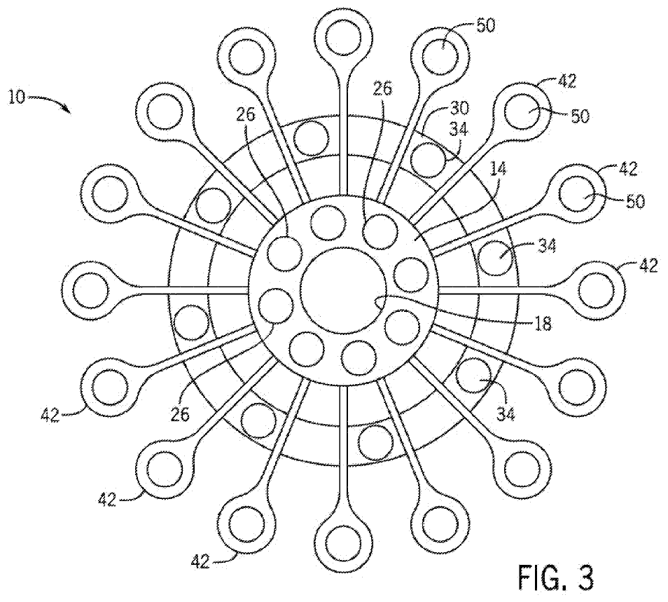

Central catheter—With reference to FIG. 1, an applicator 10 for interstitial brachytherapy is illustrated. The applicator 10 described herein advantageously enables targeted delivery of catheters under direct visualization. The applicator 10 includes a cylinder 14 with a central passageway 18 (e.g., for passage of a tandem 22 (FIG. 2), and a plurality of channels 26 (e.g., for passage of brachytherapy catheters). As such, the central cylinder 14 is a multichannel cylinder. In the illustrated embodiment, the channels 26 are spaced circumferentially around the central passageway 18 (FIG. 3). In some embodiments, the channels 26 are equally spaced circumferentially around the central passageway 18. In some embodiment, the plurality of channels 26 includes eight channels. In some embodiments, the plurality of channels 26 includes six channels. In some embodiments, the plurality of channels 26 includes any number of suitable channels.

Ring—With continued reference to FIG. 1, the applicator 10 includes a ring 30 with a plurality of outlets 34 (e.g., apertures) formed in the ring 30. The outlets 34 of the ring 30 allows passage of catheters therethrough. A plurality of connection tubes 38 extend between the cylinder 14 and the ring 30. In other words, the ring 30 is coupled to the cylinder 14 with the plurality of connection tubes 38. In the illustrated embodiment, the connection tubes 38 extend between one of the channels 26 and one of the outlets 34 on the ring 30. The connection tubes 38 are mildly curved tubes that also allow the passage of catheters.

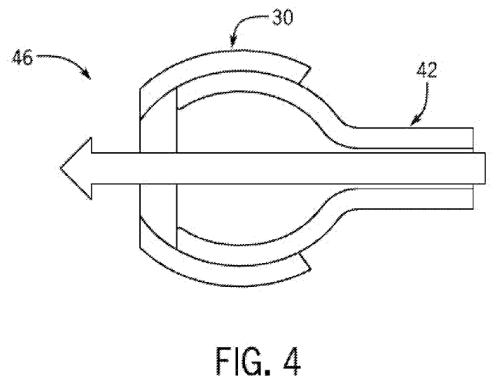

Frames—With continued reference to FIG. 1, the applicator 10 includes a plurality of frames 42. Only two of the plurality of frames 42 are illustrated in FIG. 1 for clarity. In some embodiments, the frames 42 are equally spaced apart circumferentially around the cylinder 14. The frames 42 are coupled to the ring 30 in such a way that allows movement of the frames 42. In some embodiment, the frames 42 are movable to align with the radius of the ring 30 (e.g., aimed towards the center of the ring 30). In some embodiments, each of the frames 42 is coupled to the ring 30 with a socket connection 46 (FIG. 4). The attachment of the frame 42 to the ring 30 can use a snap and fit connection or a hinge connection.

Frame channels—Each of the frames 42 includes a frame channel 50, at least one frame opening 54, a proximal end 58, and a distal end 62. In the illustrated embodiment, the distal end 62 is coupled to one of the outlets 34 on the ring 30. The frame channel 50 is for passage of catheters and/or needles received through the proximal end 58.

Laterality of Frame Channels—With reference to FIG. 2, a cross-sectional view of the applicator 10 illustrates the frame channels 50 are at least partially positioned radially outward of the ring 30 and the cylinder 14.

Wedge—In some embodiments, a wedge 66 (FIG. 1) is positioned at one of the frame openings 54. The wedge 66 guides the exit of the catheter from the frame opening 54 at different positions. In some embodiments, individual frames 42 are removable and replaced with a different frame having a frame opening 54 at a different location along the frame.

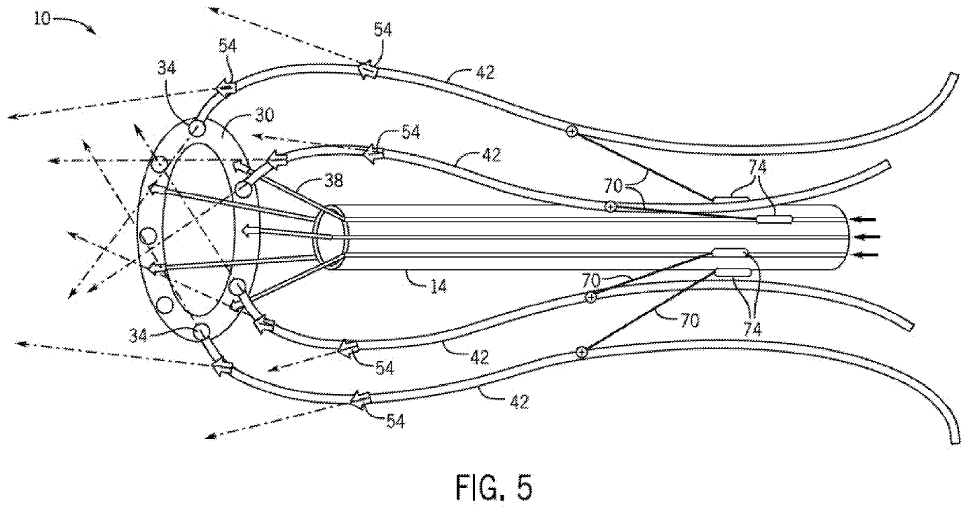

Hinge supports—With continued reference to FIG. 5, a plurality of supports 70 extend between the cylinder 14 and the frames 42. In the illustrated embodiment, each support 70 is coupled to one of the frames 42 with a hinge. Each support 70 is coupled to the cylinder 14 with an adjustable lock 74. The adjustable locks 74 are movable along a longitudinal axis 78 of the cylinder 14. In other words, the adjustable locks 74 are each individually movable along the length of the cylinder 14. In some embodiments, the adjustable locks 74 are automatic lock mechanisms, such as that disclosed in U.S. Pat. No. 5,269,063, which is incorporated herein in its entirety.

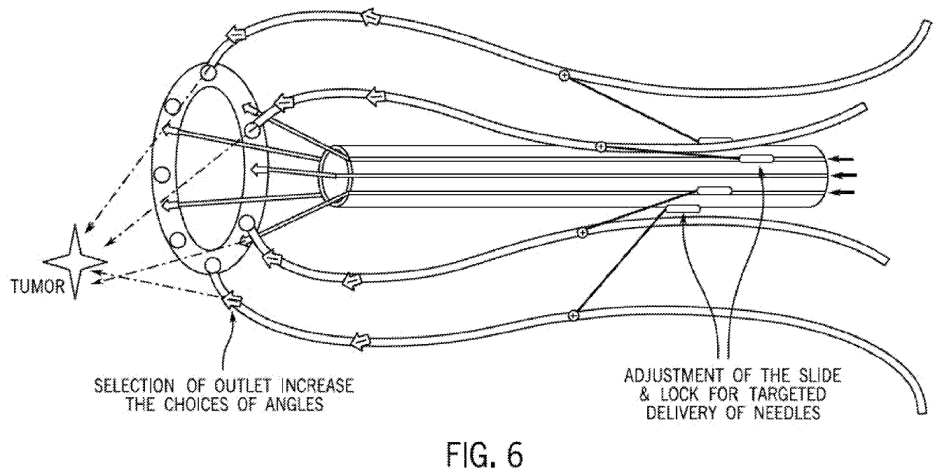

Lock—Each of the plurality of frames 42 is adjustable with respect to the cylinder 14 in response to movement of the corresponding adjustable lock 74 with respect to the cylinder. Sliding the adjustable lock 74 toward the ring 30 will cause adduction of the frame 42 and changes the angle of the frame opening 54 and the distal end 62. With reference to FIG. 6, each frame 42 has a corresponding adjustable lock 74 that is individually adjustable to align the corresponding outlets toward a desired target (e.g., tumor).

The applicator 10 detailed herein is designed based on the human anatomy of the vaginal canal to facilitate stabilization of the applicator while also fitting efficiently into the clinical workflow. A system disclosed herein includes the applicator 10, the tandem 22, and one or more brachytherapy catheters.

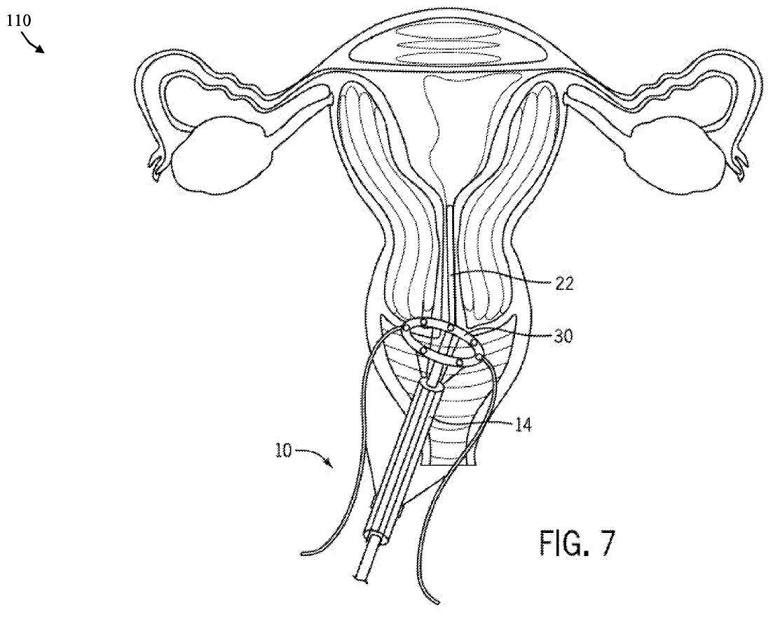

In operation, the adjustable locks 74 are moved away from the ring 30, which causes the adduction of the frames 42 to decrease the cross-sectional radius. In other words, moving the adjustable locks 74 away from the ring 30 decreases the overall cross-sectional size of the applicator 10. The applicator 10 is then inserted into the vaginal canal, and the frames 42 are abducted by moving the adjustable locks 74 toward the ring 30. In other words, moving the adjustable clocks 74 toward the ring 30 increases the distance the frame 42 extends from the cylinder 14. If indicated, the physician can place the tandem 22 via the central passageway 18 with visualization of the cervix (FIG. 7). If indicated, physicians can place tattoos that can indicate the localization of specific frame locations on the vaginal wall. If indicated, the physician can insert a brachytherapy catheter via a pre-decided channel in either the frame 42 (e.g., the frame channel 54) or the cylinder 14 (e.g., the central passageway 18 or channels 26). After placement of the needles, the applicator 10 is secured in place with sutures or external fixators.

In some embodiments, the frame along the vaginal wall has an adjustable angel via the ‘snap and fit’ mechanism (FIG. 4). In some embodiments, angles of the needle insertion are further adjustable by choosing the different outlets, either via the ring or the via the outlets in the frame.

The applicator 10 described herein has several advantages including, but not limited to: (1) direct visualization of all components of the applicator 10, the tandem 22, and then the needles; (2) no packing (e.g., gauze) is required; (3) enables targeted delivery of needles by changes in the location of the adjustable lock and/or by choosing a different exit or angles for the needle position; (4) channels can be guided along distal extent of vaginal wall; (5) may not need operation room for placement of needles; (6) the needles can be angled divergently or convergently; (7) will decrease the total amount of procedure time; (8) may increase the conformality of the radiation dosage distribution in the lesion. The applicator 10 detailed herein enables visualization for targeted delivery of interstitial implants.

In some embodiments, the applicator 10 detailed herein is used for gynecological cancers of humans or animals (e.g., uterine cancer, cervical cancer; vaginal cancer, etc.). In some embodiment, the applicator is used for other types of cancers including, for example, anal cancers.

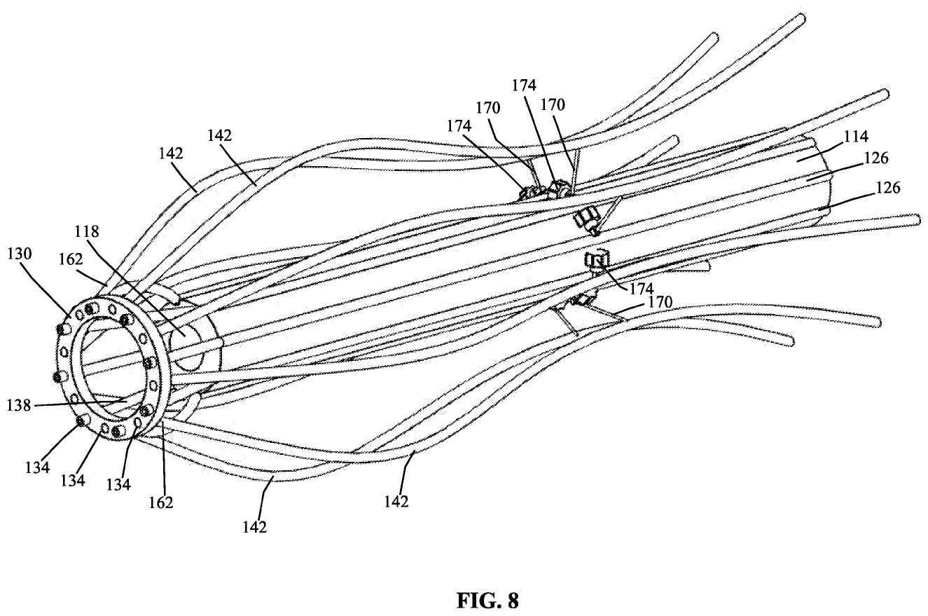

With reference to FIG. 8, an applicator 110 includes a central cylinder 114 including a passageway 118 and a plurality of channels 126 spaced circumferential around the central passageway 118. The applicator 110 further includes a ring 130 with a plurality of outlets 134, and a plurality of connection tubes 138 coupled to the ring 130. In some embodiments, each of the connection tubes 138 extends between one of the plurality of channels 126 and one of the plurality of outlets 134.

The applicator 110 a plurality of frames 142 coupled to the ring 130. Each of the frames 142 includes a distal end 162 coupled to the ring 130. As detailed herein, the frames 142 have an adjustable shape that facilitates contouring to human anatomy. The applicator 110 further includes a plurality of supports 170 extending between the cylinder 114 and the frames 142. Each of the supports 170 is coupled to the cylinder 114 with an adjustable lock 174. In the illustrated embodiment, the adjustable lock 174 is a knob gear that translates along the cylinder 114 in response to rotation of the knob gear. Each of the frames 142 is adjustable with respect to the cylinder 114 in response to movement of the corresponding adjustable lock 174 with respect to the cylinder 114.

The brachytherapy applicator disclosed herein is designed with a focus on functionality, precision, and compatibility with human anatomy. The applicator is equipped with multiple supports that extend between the cylinder and the frames. Each of these supports is paired with an adjustable lock. These locks offer a dual advantage: not only do they securely fasten the frames to the cylinder, but they also allow each frame to be individually adjusted with respect to the cylinder.

The individually adjustable frames ensure that each frame can be precisely aligned, allowing medical professionals to direct the radioactive source accurately toward the desired target, such as a tumor. This targeted approach optimizes the treatment, ensuring that the tumor receives the requisite radiation while minimizing the exposure of surrounding healthy tissues.

The applicator design disclosed herein takes into consideration the human anatomy of the vaginal canal. Advantageously, the applicator facilitates precise radiation delivery and is also anatomically compatible, providing stabilization during the procedure. Moreover, the applicator facilitates seamless integration into clinical workflows, making it an efficient tool for healthcare professionals.

In one embodiment, the applicator comprises a central cylinder including a passageway and a plurality of channels spaced circumferentially around the central passageway; a ring with a plurality of outlets; and a plurality of frames coupled to the ring. The applicator further includes a plurality of supports extending between the cylinder and the plurality of frames. Each of the plurality of supports is coupled to the cylinder with an adjustable lock; and wherein each of the plurality of frames is adjustable with respect to the cylinder in response to movement of the corresponding adjustable lock with respect to the cylinder.

Various features and advantages are set forth in the following claims.

What is claimed is:

1. A device comprising:
a central cylinder including a passageway and a plurality of channels spaced circumferentially around the central passageway;
a ring with a plurality of outlets;
a plurality of frames coupled to the ring;
a plurality of supports extending between the cylinder and the plurality of frames; wherein each of the plurality of supports is coupled to the cylinder with an adjustable lock; and wherein each of the plurality of frames is adjustable with respect to the cylinder in response to movement of the corresponding adjustable lock with respect to the cylinder.

2. The device of claim 1, wherein each of the plurality of supports is coupled to one of the plurality of frames with a hinge.

3. The device of claim 1, wherein each of the plurality of frames is equally spaced apart circumferentially around the cylinder.

4. The device of claim 1, wherein each of the plurality of frames is coupled to the ring with a socket connection.

5. The device of claim 1, further including a wedge positioned at a frame opening of at least one of the plurality of frames.

6. The device of claim 1, further including a plurality of connection tubes extending between the cylinder and the ring.

7. The device of claim 6, wherein each of the plurality of connection tubes extending between one of the plurality of channels and one of the plurality of outlets.

8. The device of claim 1, wherein the cylinder defines a longitudinal axis and the adjustable lock slides along the longitudinal axis.

9. The device of claim 1, wherein the adjustable lock is a knob gear.

10. The device of claim 1, wherein each of the plurality of frames includes a frame channel, a frame opening, and a distal end coupled to one of the plurality of outlets.

11. A system comprising:
a device of claim 1;
a tandem; and
a plurality of brachytherapy catheters.
